(19) United States
(12) Patent Application Publication
(10) Pub. No.: US 2025/0327051 A1
(43) Pub. Date: Oct. 23, 2025

(54) PROCESS FOR PRODUCING CELLULOLYTIC AND/OR HEMICELLULOLYTIC ENZYMES

(71) Applicant: IFP ENERGIES NOUVELLES, Rueil-Malmaison (FR)

(72) Inventors: Mohamed Fadhel BEN CHAABANE, Rueil-Malmaison Cedex (FR); Etienne JOURDIER, Rueil-Malmaison Cedex (FR); Celine COHEN, Rueil-Malmaison Cedex (FR); Bernard CHAUSSEPIED, Rueil-Malmaison Cedex (FR)

(73) Assignee: IFP ENERGIES NOUVELLES, Rueil-Malmaison (FR)

(21) Appl. No.: 18/851,734

(22) PCT Filed: Mar. 16, 2023
(86) PCT No.: PCT/EP2023/056825

§ 371 Date: Sep. 27, 2024

(30) Foreign Application Priority Data
Mar. 30, 2022 (FR) 2202884

(51) Int. Cl. C12N 9/24; C12N 1/14; C12N 9/42; C12R 1/885

(52) U.S. Cl. CPC C12N 9/2477; C12N 1/145; C12N 9/2437; C12R 2001/885

(57) ABSTRACT

The present invention relates to a process for producing cellulolytic and/or hemicellulolytic enzymes by a cellulolytic and/or hemicellulolytic microorganism, said process comprising at least:
a) a phase of growing the microorganism in the presence of at least one carbon-based substrate, followed by
b) a phase of producing the enzymes in the presence of at least one inducing substrate,
   and also:
c) a phase of preparing a carbon-based substrate comprising glucose and/or fructose, which carbon-based substrate is used in either and/or both of the growth phase a) and production phase b), said preparation phase comprising a step c1) of hydrolysis in an acidic aqueous medium, of sucrose to glucose and fructose.

### TECHNICAL FIELD

The present invention relates to the production of cellulolytic and/or hemicellulolytic enzymes, notably in the context of the production of sugars from cellulosic or lignocellulosic materials involving an enzymatic hydrolysis of these materials. The sugars can be used/upgraded as they are, or continue their conversion to alcohol, notably to ethanol, by fermentation.

### PRIOR ART

Since the 1970s, the transformation of lignocellulosic materials into ethanol, after hydrolysis of the constituent polysaccharides into fermentable sugars, has been the subject of very many studies. Mention may be made, for example, of the reference studies by the National Renewable Energy Laboratory (Process Design and Economics for Biochemical Conversion of Lignocellulosic Biomass to Ethanol, Humbird et al., NREL/TP-5100-57764, May 2011).

Lignocellulosic materials are cellulosic materials, i.e. materials consisting to more than 90% by weight of cellulose, and/or are lignocellulosic materials, i.e. materials consisting of cellulose, hemicelluloses, which are polysaccharides essentially consisting of pentoses and hexoses, and also lignin, which is a macromolecule of complex structure and of high molecular weight, based on phenolic compounds. For the sake of brevity, they will be grouped together in the present text under the generic term “biomass”.

Wood, straw and corn cobs are the lignocellulosic materials most commonly used, but other resources, dedicated forestry crops, residues from alcohol-yielding, sugar-yielding and cereal plants, products and residues from the paper industry and products from the transformation of lignocellulosic materials are usable. They are for the majority constituted of about 35% to 50% of cellulose, 20% to 30% of hemicellulose and 15% to 25% of lignin.

The process for the biochemical conversion of the lignocellulosic material into 2G sugary liquors notably comprises a physicochemical pretreatment step, followed by a step of enzymatic hydrolysis using an enzyme cocktail. It may be followed by a step of ethanolic fermentation of the sugars released, the ethanolic fermentation and the enzymatic hydrolysis possibly being conducted simultaneously, and a step of purification of the ethanol. One example of such a process converting biomass into ethanol is described in patent EP 3 484 945, to which reference can be made for further details.

The enzyme cocktail used for the hydrolysis is a mixture of cellulolytic enzymes (also known as cellulases) and/or hemicellulolytic enzymes. Cellulolytic enzymes have three major types of activities: endoglucanases, exoglucanases and cellobiases, the latter also being known as β-glucosidases. Hemicellulolytic enzymes notably have xylanase activities.

Enzymatic hydrolysis is efficient and is performed under mild conditions. However, the cost of producing the enzymes remains very high, possibly representing at least 20% of the cost of converting biomass to ethanol for example. As a result, numerous studies have been conducted to reduce this cost: first, optimization of the production of enzymes, by selecting hyper-productive microorganisms and by improving the processes for producing said enzymes, reduction of the amount of enzymes subsequently in hydrolysis, by optimizing the pretreatment step, by improving the specific activity of these enzymes, and by optimizing the implementation of the enzymatic hydrolysis step.

The most used cellulolytic microorganism for the industrial production of the enzyme cocktail is the fungus Trichoderma reesei. The wild-type strains have the faculty of excreting, in the presence of a carbon-based inducing substrate, for example cellulose, the enzyme cocktail considered as being the best suited for the hydrolysis of cellulose. Other proteins possessing properties vital for the hydrolysis of lignocellulosic materials are also produced by Trichoderma reesei, xylanases for example. The presence of a carbon-based inducing substrate is essential for the expression of the cellulolytic and/or hemicellulolytic enzymes. The nature of the carbon-based substrate has a strong influence on the composition of the enzyme cocktail. This is the case for xylose, which, when combined with a carbon-based inducing substrate such as cellulose or lactose, makes it possible to significantly improve the activity referred to as xylanase.

Recombinant strains have been obtained from strains of Trichoderma reesei Qm9414 (Mandels M. (1975). Microbial sources of cellulase. Biotechnol. Bioeng. 5, 81-105), RutC30 (Montenecourt, B. S. and Eveleigh, D. E., Appl. Environ. Microbiol. 1977, 34, 777-782) and CL847 (Durand et al., 1984, Proc. Colloque SFM “Génétique des microorganismes industriels” [Genetics of industrial microorganisms]. Paris. H. HESLOT Ed, pp 39-50) by cloning heterologous genes, invertase from Aspergillus niger for example, in order to diversify the source of carbon needed for the production of cellulases and/or overexpressing β-glucosidase in order to improve the enzymatic hydrolysis yield, β-glucosidases being considered to be the limiting enzymes in the reaction. These strains have retained their hyperproductivity and their ability to be cultured in a fermenter.

The incorporation of invertase in Trichoderma gives it the property of being able to consume sucrose, and therefore effluents comprising sugary liquors obtained from sugar beet or sugarcane washings, and/or molasses from a sugar refinery, for which the cost is lower and/or the availability is higher. This is what was verified, for example in patent EP 2 222 865.

Lactose and glucose remain, in an industrial enzyme cocktail production process, the most suitable carbon-based substrates. However, their cost, notably that of glucose, is high and is liable to vary significantly. The enzyme cocktail production process is also dependent on external sources of carbon.

Therefore, the use of carbon-based substrates obtained from the process for biochemical conversion of lignocellulosic materials is an advantageous pathway, and thus patent WO 2013/190214 provided an enzyme production process comprising a phase of growing the microorganisms in the presence of a carbon-based growth substrate, then a phase of producing the enzymes with a carbon-based inducing substrate in the form of liquid residue obtained from a pretreatment of lignocellulosic material comprising C5 sugars.

Current economic logic dictates that the sites for producing second-generation biofuels be the same as those for first-generation production, the assembly constituting a “biorefinery” where all of the plant material is upgraded. Thus, by starting from a sugar-producing plant, it is sought to upgrade the sugarcane and the lignocellulosic cane residues, and effluents containing sucrose may be available on site.

However, this is not the only pathway for improvement possible in enzyme production, and it may have certain drawbacks, or at the very least limitations in its application. Thus, it generally requires therefore that enzyme production take place in the biomass conversion plant, which is not always the case that is encountered/envisaged. Next, it draws off a portion of the sugars, which is intended, in the biomass conversion process, to be upgraded/converted additionally. Finally, this sugary liquid may not be sufficient to ensure, by itself, both the growth of the fungus and the enzyme production thereof. These sugars must be supplemented, notably by an inducing substrate such as lactose, in the enzyme production phase.

The objective of the invention is then to improve an enzyme production process, notably by overcoming the abovementioned drawbacks, by seeking notably to simplify the process, to reduce the costs thereof, and to make it more flexible in its industrial implementation.

### SUMMARY OF THE INVENTION

A first subject of the invention is a process for producing cellulolytic and/or hemicellulolytic enzymes by a cellulolytic and/or hemicellulolytic microorganism, said process comprising at least:
a) a phase of growing the microorganism in the presence of at least one carbon-based substrate, followed by
b) a phase of producing the enzymes in the presence of at least one inducing substrate, said process also comprising:
c) a phase of preparing a carbon-based substrate comprising glucose and/or fructose, which carbon-based substrate is used in either and/or both of the growth phase a) and production phase b), said preparation phase comprising a step c1) of hydrolysis in an acidic aqueous medium, of sucrose to glucose and fructose.

The invention has sought to exploit a source of carbon-based substrate, sucrose, in order to replace glucose, since sucrose is a less expensive compound with a more stable price than glucose, and is similar in its sugar properties. Specifically, sucrose is a disaccharide composed of glucose and fructose molecules combined via a glycosidique bond. But simply replacing glucose with sucrose is not sufficient: this is because sucrose is not assimilable in its current form by microorganisms of the filamentous fungi type Trichoderma, as these microorganisms are not naturally endowed with the invertase gene.

A first solution may consist in genetically modifying the microorganisms in order to provide them with this gene. The publication by Lucas Miranda Fonseca et al. “Rational engineering of the Trichoderma reesei RUT-C30 strain into an industrially relevant platform for cellulase production” published in the journal Biotechnology for Biofuels (2020) thus proposes to introduce several genetic modifications in the RUT-C30 strain of T. reesei, notably enabling the expression of the invertase gene, and therefore making it possible to use, at least in part, sucrose as carbon-based substrate in the process for producing enzymes from this modified strain. Although this targeted genetic modification technique is effective, it is complex to implement, and must be repeated whenever it is desired to change strain.

The invention has therefore consisted not in modifying the strains, but in modifying the sucrose to make it assimilable. This modification is an acid hydrolysis, which makes it possible to cleave the glycosidic bond of the molecule so that it breaks down to glucose and fructose, both of which are assimilable by the microorganisms targeted by the invention.

Advantageously, the cellulolytic and/or hemicellulolytic microorganism of interest for the invention is chosen from microorganisms devoid of the invertase gene and capable of consuming glucose and/or fructose: use can therefore be made in the invention of unmodified strains or, more specifically, strains that have not been modified so as to make them capable of assimilating sucrose. The microorganisms targeted by the invention are preferably microorganisms of fungus type, notably of filatamentous fungus type.

The cellulolytic and/or hemicellulolytic microorganism may be chosen in order to implement the invention from the strains of fungi belonging to the genera Trichoderma, Aspergillus, Penicillium or Schizophyllum, and belonging preferably to the species Trichoderma reesei.

Advantageously, on completion of the preparation phase c), an aqueous solution comprising glucose and fructose is obtained which can be used directly or after any concentration-adjusting operations (or other treatment of filtration, etc. type), as carbon-based substrate in the growth step a) and/or production step b).

The hydrolysis may be complete or partial: in the latter case, a (small) amount of sucrose May still be present in the solution obtained at the end of hydrolysis.

According to one embodiment, at least one inducing substrate, notably lactose, can be added to the aqueous solution comprising glucose and fructose obtained on conclusion of the preparation phase c), in order to use said solution as a substrate in the production step b). Thus, a solution of carbon-based substrate which is ready-to-use for the production step b) with a mixture of inducing and non-inducing substrates is obtained. It may also be used for step a), it being possible for the lactose to also be used as a carbon-based growth substrate. In this case, there is only a single solution supplying the bioreactor for both steps, which simplifies its preparation, its storage and its method of introduction into the bioreactor.

It should be noted that the process according to the invention may carry out the growth step a) and production step b) in the same bioreactor. Alternatively, it may be carried out with the first bioreactor dedicated to the growth step a), and a second bioreactor dedicated to the production step b).

According to another embodiment, at least one inducing substrate, notably lactose, is added to the acidic aqueous medium during the sucrose hydrolysis step c1). The advantage is that lactose is a disaccharide, like sucrose, but which is the combination of a glucose and a galactose via a glycosidic bond, which is assimilable by the fungal strains targeted by the invention. However, the inventors have demonstrated that lactose is significantly more difficult to hydrolyze under acid conditions than sucrose. All the same, a small portion of lactose would be hydrolyzed, the glucose and galactose resulting from its hydrolysis are also assimilable by the strains.

This embodiment can be combined with the preceding one, by adding the inducing substrate partly during the hydrolysis, and partly at the end of the hydrolysis.

It should also be noted that at the end of the hydrolysis, the aqueous solution obtained can be used immediately, in the enzyme production plant. It can also be stored for later use, and the hydrolysis can advantageously be continued during the storage if need be, generally at ambient temperature, the solution preferably being maintained at an acid pH until it is used, in order to limit the risks of contamination.

According to a first variant, the sucrose hydrolysis step c1) can be carried out in an acidic aqueous medium at a pH below or equal to 5, notably below or equal to 4, notably below or equal to 3.5 or 3, preferably between 1 and 3.

In order to obtain a hydrolysis medium with acid pH, a strong acid, of hydrochloric acid or sulfuric acid type, is added to an aqueous solution in a sufficient amount to adjust the pH of the solution to the chosen value.

Alternatively, it is possible to add a weak acid, of organic acid type.

Yet another alternative may be to acidify the aqueous solution by introducing thereinto carbon dioxide CO₂ in gaseous form (bubbling): this is in fact a (weak) acid and it is obtained from the fermentation of the sugars to alcohol in the biomass-to-alcohol conversion process. It is therefore available if the enzyme production is carried out at the biomass conversion site.

It is also possible to combine together at least two of these various alternatives: addition to an aqueous solution of at least one strong acid, of at least one weak acid, introduction of CO₂.

Advantageously, notably with this first variant, the sucrose hydrolysis step c1) is carried out in an acidic aqueous medium at a temperature of at least 110° C., notably between 110° C. and 125° C., notably between 110° C. and 120° C., in order to also ensure the sterilization of said medium. Specifically, the hydrolysis is accelerated if the medium in which the hydrolysis is carried out is heated, but not necessarily at such high temperatures. But performing the hydrolysis at at least 110° C. makes it possible to simultaneously sterilize the solution, which is necessary for its introduction into the enzyme production process. It is then advantageous, as mentioned above, to add one or more inducing substrates, such as lactose, to this medium before sterilization, which makes it possible to introduce the sterile solution comprising all the necessary substrates into the bioreactor (and which therefore avoids having to carry out a separate sterilization of the inducing substrates).

In a second variant, the sucrose hydrolysis step c1) is carried out in an acidic aqueous medium comprising at least in part, notably solely, an acidic hemicellulosic hydrolysate.

Advantageously, this hemicellulosic hydrolysate is obtained from an acid pretreatment step of a lignocellulosic biomass, and it comprises monomers and oligomers of predominantly C5 sugars. Unlike the abovementioned patent WO 2013/1900214, this hydrolysate is not used here directly as carbon-based substrate, but firstly as offering a (strongly) acidic medium for the prior hydrolysis of the sucrose. It should be noted that this hydrolysate is acidic, since the biomass pretreatment from which it is obtained preferably comprises a step of acid impregnation, an example of which is described in the abovementioned patent EP 3 484 945.

Thus, ultimately, a solution is obtained containing the C5 sugars of the hydrolysate, which are assimilable by the strains (microorganisms), and glucose and fructose obtained from the hydrolysis of the sucrose (and optionally also non-hydrolyzed sucrose). This variant is very advantageous owing to its flexibility: it is possible to meter the proportion of C5 sugars and the proportion of glucose/fructose of the solution obtained in the end, by metering the amount of hydrolysate and the amount of sucrose added thereto, as a function of the availability of both of the carbon sources.

There is another advantage to using a hydrolysate as acidic medium for hydrolysis of the sucrose: it is possible to carry out the hydrolysis by heating the solution, in order to accelerate it/boost it, but up to temperatures lower than in the case of the first variant: thus, the hydrolysis step c1) can be carried out at a temperature below or equal to 100° C., notably below or equal to 80° C., notably between 30° C. and 60° C., since it is no longer necessary to sterilize the solution obtained before using it.

This is because the hemicellulosic hydrolysates are acidic media, which contain sugars, and also inhibitors generated during the acid pretreatment of the biomass, such as acetic acid, furfural, 5-HMF or else phenolic compounds, which greatly limits the risks of contamination.

It is also possible to combine the two variants, by using, as acidic hydrolysis medium, a hemicellulosic hydrolysate which is optionally diluted, concentrated, etc., by adding an acid or CO₂ thereto.

It is seen that it is possible to adjust as best possible the degree of hydrolysis of the sucrose during step c1) by acting on the operating conditions, namely by adjusting the pH of the medium to a more or less acidic pH (by addition of acid and/or hydrolysate at variable concentration) and/or by adjusting the temperature at which the hydrolysis is carried out and/or by adjusting the hydrolysis time.

According to the invention, it is possible to select various operating conditions, notably depending on the chosen variant. The temperature at which the hydrolysis is performed, and also the pH of the medium in which it is performed and the hydrolysis time are the most significant operating conditions. Generally, it was observed that the hydrolysis takes place more rapidly, the higher the temperature and the lower the pH.

According to a first embodiment, the sucrose hydrolysis step c1) can be carried out in an acidic aqueous medium at a temperature of at least 110° C., at a pH of at most 5 and over a time preferably of between 10 minutes and 10 hours. It is favored for the implementation of the first variant, where it is necessary to sterilize the medium, or when a (very) rapid hydrolysis is desired. And it is noted that in this case, where the temperature is high, it is possible to adopt a slightly less acidic pH than with a lower temperature (when not looking to sterilize).

According to a second embodiment, the sucrose hydrolysis step c1) can be carried out in an acidic aqueous medium at a temperature of at most 100° C., notably between 30° C. and 60° C., at a pH of at most 4, and over a time preferably of between 1 hour and 24 hours. It is favored for the implementation of the second variant, notably in the case where it is desired to have a relatively fast hydrolysis time.

According to a third embodiment, the sucrose hydrolysis step c1) can be carried out in an acidic aqueous medium at a temperature of between 10° C. and 30° C., at a pH of at most 4 and over a time preferably of more than 24 hours. It is favored for the implementation of the second variant, notably in the case where the hydrolysis may be carried out over a long period of time. Here, it is not therefore necessary to heat the hydrolysis medium. This is an advantageous embodiment when there is a sucrose storage tank in which the hydrolysis can be left to take place until use, at ambient temperature (by feeding the bioreactor where the enzyme production takes place).

### LIST OF FIGURES

FIG. 1 represents the change in the concentration of biomass (in the following examples, these are microorganisms in the form of fungi) over time in an enzyme production process with various solutions of carbon-based substrate, (on the x-axis, the time in hours, on the y-axis, the amount of biomass in g/l).

FIG. 2 represents the change in the concentration of enzymes over time in an enzyme production process with various solutions of carbon-based substrate, (on the x-axis, the time in hours, on the y-axis, the amount of enzymes in g/l).

FIG. 3 represents the change in the concentration of sucrose over time: on the x-axis, the time in hours, on the left y-axis of the graph, the estimate of the amount of sucrose in the fed-batch solution in g/l, and on the right y-axis the amount of sucrose in the bioreator where both the microorganism growth step and the enzyme productions are carried out, in g/l.

### DESCRIPTION OF THE EMBODIMENTS

### Tests of Sucrose Hydrolysis in an Acidic Medium

Firstly, preliminary hydrolysis tests of sucrose and lactose (by way of comparison) were carried out in aqueous solutions of sulfuric acid at various pH values and various temperatures, according to the first variant of the invention.

The operating procedure is the following:

3 solutions of around 220 g/l are prepared with:
100% sucrose: solution referred to as “pure sucrose” in the following tables
100% lactose: solution referred to as “pure lactose” in the following tables
75% sucrose+25% by weight lactose: solution referred to as “as a mixture” in the following tables

The solutions are separated into four, and the pH is adjusted with sulfuric acid, targeting the following four pH values: 1.5; 2.0; 2.5; 3.0.

An aliquot is taken and then each of the 4 solutions is separated into two in order to test two sterilization conditions: (here the sterilization takes place at the same time as the hydrolysis of the disaccharides).
conditions (1): 110° C. for 35 minutes
conditions (2): 121° C. for 20 minutes

The results are as follows:

After sterilization, a coloration of the solutions is observed visually for pH values below 2.0, whether with sucrose or with lactose, and regardless of the condition (1) or (2) chosen.

The residual sugars are assayed after sterilization/hydrolysis by high-performance liquid chromatography (HPLC).

These results are given in detail in the two tables below:
table 1 indicates the percentage hydrolysis of sucrose for the various solutions, depending on whether they were sterilized according to condition (1) or (2),
table 2 indicates the percentage hydrolysis of lactose, in a comparable manner to table 1.

TABLE 1

| | % hydrolysis of sucrose | | | |
|---|---|---|---|---|
| | Conditions (2) | | Conditions (1) | |
| pH | Pure sucrose | as a mixture | Pure sucrose | as a mixture |
| 1.5 | 99% | 98% | 100% | 95% |
| 2 | 100% | 98% | 98% | 100% |
| 2.5 | 100% | 100% | 95% | 95% |
| 3 | 100% | 97% | 88% | 88% |

TABLE 2

| | % hydrolysis of lactose | | | |
|---|---|---|---|---|
| | Conditions (2) | | Conditions (1) | |
| pH | Pure lactose | as a mixture | Pure lactose | as a mixture |
| 1.5 | 72% | 68% | 26% | 31% |
| 2 | 24% | 22% | 12% | 15% |
| 2.5 | 0% | 6% | 0% | 7% |
| 3 | 0% | 11% | 0% | 4% |

It is seen that:
at 121° C., with conditions (2), the sucrose is completely hydrolyzed to glucose+fructose, regardless of the pH from among the four pH values tested,
at 110° C., with conditions (1), the sucrose is completely hydrolyzed, when the pH is below or equal to 2.5.

These tests therefore demonstrate that it is possible to completely or almost completely hydrolyze sucrose in an acidic medium, by choosing a strongly acidic pH (at most 5, preferably at most 4, preferably at most 3.5 or 3), a high temperature making it possible to carry out the sterilization at the same time, and this being in a time of 20 minutes, therefore rapidly.

Is also seen that the hydrolysis of lactose does not take place or barely takes place, unless a very acidic pH, of at most 2, is adopted.

### EXAMPLES

Firstly, the enzyme production process used to illustrate the invention is described with the aid of the examples that follow.

These examples relate to the second variant of the invention with hydrolysis of the sucrose by a hemicellulosic hydrolysate (also referred to as “C5 sugar liquor” or “C5 liquor”).

### The Strain

The strain of fungus used in the following examples is described in patent WO2015/193587: it is the variant referenced 130G9 (SEQ ID No. 7 as nucleic acid, SEQ ID No. 8 as polypeptide in table 1 of that patent) of Trichoderma reesei. It will be denoted hereinafter by the term “the strain”. It does not possess the invertase gene and does not have the ability to assimilate sucrose.

### The Preculture Phase

The strain preculture phase is carried out in the following manner:

The strain is precultured in a Fernbach flask for 72 h at 30° C. and under stirring at a stirring speed of 150 rpm.

### Composition of the Preculture Medium:

### In a Fernbach Flask:

62.5 ml of 4N medium (composition given below)
1.25 g of dipotassium phthalate
1 g of “corn steep” (maize proteins), commercially available from Roquette under the trade name Solulys.
quantity sufficient: 200 ml of distilled water

The mineral medium (referred to as “4N medium”) has the following composition: KOH 1.66 g/l, 85% H₃PO₄ 2 ml/l, (NH₄)₂SO₄ 2.8 g/l, MgSO₄, 7 H₂O 0.6 g/l, CaCl₂0.6 g/l, MnSO₄ 3.2 mg/l, ZnSO₄, 7 H₂O 2.8 mg/l, CoCl₂ 10H₂O 4.0 mg/l, FeSO₄, 7 H₂O 10 mg/l.

The pH is adjusted to 5 with sodium hydroxide before sterilization.

### In a Separate Flask:

8.5 g of glucose in 50 ml of distilled water

### The Cultures in Bioreactors:

### The Growth Phase

The preculture is transferred to four mechanically-stirred 1.5 L bioreactors of CSTR (continuous stirred-tank reactor) type, with a seeding volume of 10% (v/v).

### Composition of the Culture Medium:

400 ml of 4N medium
320 ml of glucose at 37.5 g/l, i.e. an initial glucose concentration of 15 g/l
1 g of corn steep from Roquette
80 ml from the preculture in Fernbach flask.

The pH is maintained at 4.4 by adding a 5.5 N aqueous ammonia solution. The pO₂ (concentration of dissolved O₂ expressed relative to the saturation concentration) is regulated at 40% by stirring. The temperature is stabilized at 27° C.

### The Production Phase

After consumption of the glucose, the production phase starts by induction with the continuous addition of a sugar solution at around 400 g/kg, at a flow rate of 1.25 ml/h. The pH is regulated at 4 and the temperature at 25° C.

The fed-batch solutions are prepared by dissolving lactose and sucrose or glucose in a solution of hemicellulosic hydrolysate obtained from straw pretreated by steam explosion under acidic conditions. This hemicellulosic hydrolysate, referred to as “BHS 7.3” liquor is obtained after washing, with water, straw pretreated at 185° C. for 5 minutes after impregnation with sulfuric acid (acidity of 1.8%).

The composition of the BHS 7.3 liquor is given in table 3 below.

The acronym SC for Solids Content corresponds to the solid portion of a liquid/solid mixture which is soluble in the liquid+the solid portion which is insoluble in the liquid in question.

The acronym ISC for Insoluble Solids Content includes only the solid portion which is insoluble in the liquid/solid mixture.

TABLE 3

| SC | ISC | Glucose | Xylose | Acetic acid | HMF | Furfural |
|---|---|---|---|---|---|---|
| (%) | (%) | (g/l) | (g/l) | (g/l) | (g/l) | (g/l) |
| 9.04 | 0.08 | 10.05 | 35.07 | 1.81 | 0.58 | 0.19 |

The compositions of the solutions are described in detail below:

### Example 1: Control with Glucose+Lactose (not Sterilized)

A 1 kg solution was prepared by dissolving, in 450 g of BHS 7.3 liquor, 330 g of glucose monohydrate, 120 g of water and 100 g of lactose (lactose as inducing substrate). The dissolving is carried out in a beaker at ambient temperature using a magnetic stirrer bar. The solution obtained is not sterilized. It is the positive control.

The term “ambient temperature” is understood throughout the present text to mean a temperature usually between 10° C. and 30° C., generally between 15° C. and 25° C., and in the present case around 20° C.

### Example 2: Use of Sucrose+Lactose (without Sterilization)

A 1 kg solution was prepared by dissolving, in 450 g of BHS 7.3 liquor, 300 g of sucrose 150 g of water and 100 g of lactose. There is no heating step to hydrolyze the sucrose at acid pH. The fed-batch solution is maintained at ambient temperature. The latter will hydrolyze partially and slowly throughout the 215 hours of the experiment.

### Example 3: Heating Sucrose+C5 Liquor to Hydrolyze it then Adding Lactose to the Solution (without Sterilization)

A 900 g solution was prepared by dissolving, in 450 g of BHS 7.3 liquor, 300 g of sucrose+150 g of water, then the solution is heated at 110° C. for 20 minutes, then 100 g of lactose is added under non-sterile conditions.

### Example 4: Use of Sucrose and Lactose with Sterilization

A 1 kg solution was prepared by dissolving, in 450 g of BHS 7.3 liquor, 300 g of sucrose+150 g of water+100 g of lactose, then heating (autoclaving) of the solution is carried out at 120° C. and for 20 minutes. The solution is sterile (limits the risks of contamination) and the sucrose is hydrolyzed to glucose and fructose.

### Example 5 (Comparative): Use of Sucrose+Lactose without Acidic Hydrolysis of the Sucrose

A 1 kg solution was prepared by dissolving, in 450 g of water, 300 g of sucrose, 150 g of water and 100 g of lactose, then heating (autoclaving) of the solution is carried out at 120° C. and for 20 minutes. The solution is sterile. Therefore there is no contact here between the sucrose and an acidic medium prior to it being introduced into the bioreactor. The fed-batch solution is maintained at ambient temperature.

### The Monitoring

On samples taken daily, the following measurements were carried out:
determination of the biomass by membrane filtration,
assaying of the residual sugar by HPLC,
assaying of the extracellular enzymes by the Lowry method after precipitation with TCA and BSA standard, after separation of the microorganism by filtration or centrifugation.

The cellulolytic activities are determined by:
the filter paper activity (FPU: filter paper unit), which makes it possible to assay the overall activity of the enzyme cocktail: endoglucanases and exoglucanases,
the aryl β-glucosidase activity, for the specific activities.

The FPU activity is measured on Whatman No. 1 paper (procedure recommended by the IUPAC biotechnology commission) at the initial concentration of 50 g/l: a determination is made of the sample of the enzymatic solution to be analyzed which releases the equivalent of 2 g/l of glucose (colorimetric assay) in 60 minutes. The principle of the filter paper activity is to use a DNS (dinitrosalicylic acid) assay to determine the amount of reduced sugars obtained from a Whatman No. 1 paper.

The substrate used to determine the aryl β-glucosidase activity is p-nitrophenyl-β-D-glucopyranoside (PNPG). It is cleaved by β-glucosidase which releases p-nitrophenol. A unit of aryl β-glucosidase activity is defined as the amount of enzyme needed to produce 1 μmol of p-nitrophenol from PNPG per minute and is expressed in IU/mL.

The specific activities are obtained by dividing the activities expressed in IU/mL by the concentration of proteins. They are expressed in IU/mg. The cultures were indeed effected until the end of the experiments.

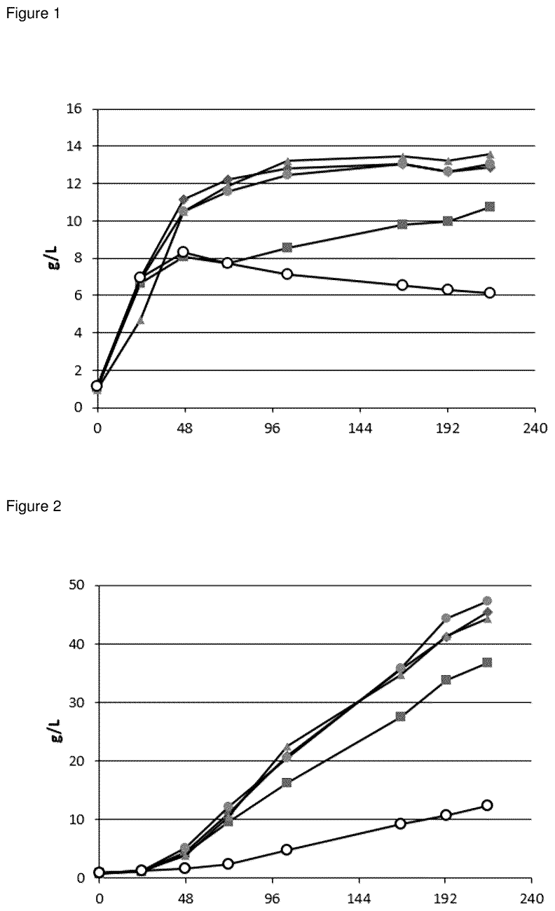

FIG. 1 represents the monitoring of the concentration in g/l of fungi in the bioreactor as a function of the time in hours. The time t=0 corresponding to the start of the process, comprising a first phase of growth of the fungi of 24 hours, with a feed from the bioreactor of carbon-based substrate (glucose as mentioned above) in one go, referred to as batch feeding, then a second phase of producing the enzymes, with a feed from the reactor of carbon-based substrate, referred to as fed-batch feeding, according to the compositions defined above (gradual feed according to a given frequency of addition). The curve with the diamonds corresponds to example 1 (control), the curve with the squares to example 2, the curve with the triangles corresponds to example 3, the curve with the filled circles to example 4, and the curve with the hollow circles to example 5 (comparative).

FIG. 2 represents the monitoring of the concentration in g/l of enzymes in the bioreactor as a function of the time in hours, the time t=0 corresponding to the start of the process, with the same conventions as for FIG. 1 (24 hours of growth, followed by production) for examples 1 to 5.

For the five fermentations, as can be seen from FIGS. 1 and 2, the enzyme production of examples 3 and 4 with sucrose hydrolyzed before the experiment is identical to that of example 1 which is the control (with glucose). As for example 5, it is seen that it results in a much lower production of fungi and enzymes than the other examples, which demonstrates that, in this case, the non-hydrolyzed sucrose is not assimilated by the fungi, and that the growth of the fungi and the enzyme production is due only to the presence of lactose in the fed-batch solution.

Regarding example 2, with sucrose hydrolyzed without heating: it produces fewer enzymes than the other examples, but however in an amount comparable therewith, which is surprising. Looking at the final assays of the residual sugar, it is seen that there is an accumulation of sucrose over time which is not consumed, which explains the lower production performance. However, if the sucrose was not hydrolyzed at all in the fed-batch solution, the final concentration of sucrose would have theoretically been equal to 86 g/l (instead of 27 g/l), since the strain tested does not possess invertase and is not therefore able to consume the sucrose.

The final assay of sugars in the fed-batch solution maintained at ambient temperature throughout the experiment, after 24 hours, in the end contains very little sucrose: 50 g/l instead of the 350 g/l added initially.

The acidic pH of the fed-batch solution enabled the hydrolysis of the sucrose, just like when the hydrolysis is carried out with heating (sterilization), but at a slower rate.

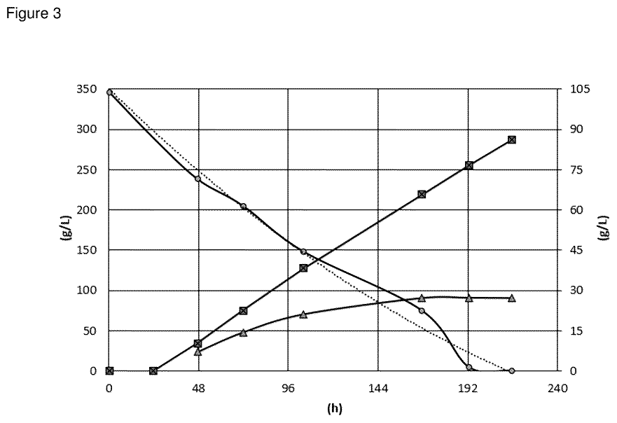

FIG. 3 represents, for this example 2, a graph with, on the x-axis, the time in hours, on the y-axis, the concentration of sucrose in the fed-batch solution (on the left of the graph) in g/l, and in the bioreactor (on the right of the graph) also in g/l, the time t=0 corresponding to the start of the process, with the same conventions as for FIGS. 1 and 2 (24 hours of growth, followed by production).

The curve with the circles corresponds to the change in concentration of sucrose in the fed-batch solution measured in its storage tank (in fluidic connection with the bioreactor in order to feed it).

The curve with the squares corresponds to the theoretical accumulation of sucrose in the bioreactor.

The curve with the triangles corresponds to the accumulation of sucrose measured in the bioreactor.

Assuming that the sucrose is hydrolyzed only in the tank containing the fed-batch solution, and not in the bioreactor which is at pH 4, and using the measurement of the accumulation of sucrose over time in the bioreactor, it is thus possible to estimate the kinetics for hydrolysis of sucrose in the fed-batch solution.

It is thus seen that, under these conditions, around one week was required in order to completely hydrolyze the sucrose in its storage tank at ambient temperature.

The specific β-glucosidase and FPU activities are indicated in table 4 below for examples 1 to 5. They are all of the same order, irrespective of the fermentation, which indicates that the enzymes produced are of equivalent quality.

TABLE 4

| | β-Glucosidase activity | FPU activity |
|---|---|---|
| Examples | (IU/mg) | (IU/mg) |
| Example 1 - Control | 26 | 0.9 |
| Example 2 | 24 | 1.1 |
| Example 3 | 25 | 0.9 |
| Example 4 | 24 | 1.0 |
| Example 5 - Comparative | 23 | 0.9 |

Table 5 below collates, for examples 1 to 5, the specific rates of enzyme production qₚ. This is calculated by dividing the protein production rate expressed in g/l/h by the concentration of microorganisms (as is known to a person skilled in the art). They are of the same order for the fermentations of examples 1 to 4, but around two times lower for example 5, which indicates that their lack of assimilable sugar has not made it possible to obtain good production performance.

TABLE 5

| | Specific production rate qₚ |
|---|---|
| Examples | (mgₚ/gₓ/h) |
| Example 1 - Control | 23 |
| Example 2 | 26 |
| Example 3 | 22 |
| Example 4 | 24 |
| Example 5 - Comparative | 12 |

HPLC analysis of the fed-batch solutions at the end of fermentation confirms that the lactose from example 4 has not been degraded (concentration=100 g/l), after autoclaving or without autoclaving, and that the sucrose is completely hydrolyzed to glucose and fructose after autoclaving, owing to the strongly acidic pH of the solution, equal to 2.9.

An additional experiment was carried out by preparing a fed-batch solution similar to that of example 2. This solution was divided into two portions: one portion incubated at 20° C. for 215 hours, and one portion incubated at 50° C. for 215 hours. It appears that after 24 h at 50° C., all the sucrose is hydrolyzed, whereas 60% of the sucrose is hydrolyzed after 215 h at 20° C.

From these various examples and results, it is understood that the invention consisting in hydrolyzing sucrose in an acidic medium makes it possible to render it assimilable by the fungal strain without having to genetically modify it in order to add the invertase gene thereto. It is also understood that this hydrolysis is promoted/accelerated by heating, either at moderate temperature, or at higher temperature, notably if it is also necessary to sterilize the substrate solution before it is brought into contact with the strain.

The invention offers great flexibility of implementation,
by using two possible types of acidic medium: made by adding acid, or obtained directly by a C5 sugar liquor obtained during the biomass conversion, and which is in fact at a sufficiently acidic pH for this hydrolysis,
and by using different operating conditions (notably the hydrolysis temperature) depending on the requirements (high temperature if sterilization is required, lower temperature or even ambient temperature if the hydrolysis can be left to take place more slowly, pH which can be chosen to be more acidic, the lower the temperature, etc.).

1. A process for producing cellulolytic and/or hemicellulolytic enzymes by a cellulolytic and/or hemicellulolytic microorganism, said process comprising at least:
a) a phase of growing the microorganism in the presence of at least one carbon-based substrate, followed by
b) a phase of producing the enzymes in the presence of at least one inducing substrate,
characterized in that said process also comprises:
c) a phase of preparing a carbon-based substrate comprising glucose and/or fructose, which carbon-based substrate is used in either and/or both of the growth phase a) and production phase b), said preparation phase comprising a step c1) of hydrolysis in an acidic aqueous medium, of sucrose to glucose and fructose,
the cellulolytic and/or hemicellulolytic microorganism being chosen from microorganisms devoid of the invertase gene and capable of consuming glucose and/or fructose, notably strains of fungi,
and the sucrose hydrolysis step c1) is carried out in an acidic aqueous medium comprising at least in part an acidic hemicellulosic hydrolysate.

2. The process as claimed in claim 1, characterized in that the cellulolytic and/or hemicellulolytic enzyme is chosen from the strains of fungi belonging to the genera Trichoderma, Aspergillus, Penicillium or Schizophyllum, and belonging preferably to the species Trichoderma reesei.

3. The process as claimed in claim 1, characterized in that, on conclusion of the preparation phase c), an aqueous solution comprising glucose and fructose is obtained, and in that use is made of said solution, directly or after any concentration-adjusting operations, as carbon-based substrate in the growth step a) and/or production step b).

4. The process as claimed in claim 1, characterized in that at least one inducing substrate, notably lactose, is added to the aqueous solution comprising glucose and fructose obtained on conclusion of the preparation phase c), notably in order to use said solution as a substrate in the production step b).

5. The process as claimed in claim 1, characterized in that at least one inducing substrate, notably lactose, is added to the acidic aqueous medium during the sucrose hydrolysis step c1).

6. The process as claimed in claim 1, characterized in that the sucrose hydrolysis step c1) is carried out in an acidic aqueous medium at a pH below or equal to 5, notably below or equal to 4, notably below or equal to 3.5 or 3, preferably between 1 and 3.

7. The process as claimed in claim 1, characterized in that the acid pH of the acidic aqueous medium of the hydrolysis step c1) is obtained by adding, to an aqueous solution, at least one strong acid and/or at least one weak acid and/or gaseous carbon dioxide, notably obtained from a fermentation.

8. The process as claimed in claim 1, characterized in that the sucrose hydrolysis step c1) is carried out in an acidic aqueous medium at a temperature of at least 110° C., notably between 110° C. and 125° C., notably between 110° C. and 120° C., in order to also ensure the sterilization of said medium.

9. The process as claimed in claim 1, characterized in that the sucrose hydrolysis step c1) is carried out in an acidic aqueous medium comprising solely an acidic hemicellulosic hydrolysate.

10. The process as claimed in claim 1, characterized in that the hemicellulosic hydrolysate is obtained from an acid pretreatment step of a lignocellulosic biomass, and in that it comprises monomers and oligomers of predominantly C5 sugars.

11. The process as claimed in claim 1, characterized in that the hydrolysis step c1) is carried out at a temperature below or equal to 100° C., notably below or equal to 80° C., notably between 30° C. and 60° C., or between 10° C. and 30° C.

12. The process as claimed in claim 1, characterized in that step c1) of hydrolysis of the sucrose is carried out in an acidic aqueous medium at a temperature of at least 110° C., at a pH of at most 5 and over a time preferably of between 10 minutes and 10 hours.

13. The process as claimed in claim 1, characterized in that step c1) of hydrolysis of the sucrose is carried out in an acidic aqueous medium at a temperature of at most 100° C., notably between 30° C. and 60° C., at a pH of at most 4, and over a time preferably of between 1 hour and 24 hours.

14. The process as claimed in claim 1, characterized in that step c1) of hydrolysis of the sucrose is carried out in an acidic aqueous medium at a temperature of between 10° C. and 30° C., at a pH of at most 4 and over a time preferably of more than 24 hours.
